(19) United States
(12) Patent Application Publication
(10) Pub. No.: US 2024/0112773 A1
(43) Pub. Date: Apr. 4, 2024

(54) Medical Record Management Assembly

(71) Applicant: George Hammitt, III, Franklin, TN (US)

(72) Inventor: George Hammitt, III, Franklin, TN (US)

(21) Appl. No.: 17/959,837

(22) Filed: Oct. 4, 2022

(51) Int. Cl. G16H 10/60; G16H 20/00; G16H 50/20; G16H 70/20

(52) U.S. Cl. CPC G16H 10/60; G16H 20/00; G16H 50/20; G16H 70/20

(57) ABSTRACT

A medical record management assembly includes a personal electronic device which stores operational software comprising a medical history of a patient to facilitate the patient to control access to their medical history. The personal electronic device is in remote communication with an extrinsic communication network. A remote data storage device is in remote communication with the extrinsic communication network thereby facilitating the remote data storage device to communicate with the personal electronic device thereby facilitating the medical history to be stored in a centralized location. The remote data storage device is in remote communication with a plurality of health care systems thereby facilitating the personal electronic device to communicate the selected portions of the medical history to the plurality of health care systems. In this way the personal electronic device can facilitate health care professionals to access the selected portions of the medical history for analysis.

### CROSS-REFERENCE TO RELATED APPLICATIONS

Not Applicable

### STATEMENT REGARDING FEDERALLY SPONSORED RESEARCH OR DEVELOPMENT

Not Applicable

### THE NAMES OF THE PARTIES TO A JOINT RESEARCH AGREEMENT

Not Applicable

### INCORPORATION-BY-REFERENCE OF MATERIAL SUBMITTED ON A COMPACT DISC OR AS A TEXT FILE VIA THE OFFICE ELECTRONIC FILING SYSTEM

Not Applicable

### STATEMENT REGARDING PRIOR DISCLOSURES BY THE INVENTOR OR JOINT INVENTOR

Not Applicable

### BACKGROUND OF THE INVENTION

### (1) Field of the Invention

The disclosure relates to medical record devices and more particularly pertains to a new medical record device for centralizing a patient's medical history. The device includes a personal electronic device and operational software stored on the personal electronic device. The device includes a remote data storage device that is in remote communication with the personal electronic device. The remote data storage device stores medical data for the owner of the personal electronic device. Furthermore, the remote data storage device releases medical history to health care facilities when the operational software receives an authorization command. The device includes an artificial intelligence unit for analyzing the patient's medical data and subsequently making recommendations for medical care.

### (2) Description of Related Art Including Information Disclosed Under 37 CFR 1.97 and 1.98

The prior art relates to medical record devices including a patient encounter electronic system that includes diagnostic templates, master databases and a graphical user interface. The prior art discloses a medical record access device which includes a personal computer, a keyboard and a machine readable questionnaire. The prior art discloses a portable medical record which includes a plurality of processors connected to a medical record database. The prior art discloses a variety of electronic means of storing and communication patient medical records.

### BRIEF SUMMARY OF THE INVENTION

An embodiment of the disclosure meets the needs presented above by generally comprising a personal electronic device which stores operational software comprising a medical history of a patient to facilitate the patient to control access to their medical history. The personal electronic device is in remote communication with an extrinsic communication network. A remote data storage device is in remote communication with the extrinsic communication network thereby facilitating the remote data storage device to communicate with the personal electronic device thereby facilitating the medical history to be stored in a centralized location. The remote data storage device is in remote communication with a plurality of health care systems thereby facilitating the personal electronic device to communicate the selected portions of the medical history to the plurality of health care systems. In this way the personal electronic device can facilitate health care professionals to access the selected portions of the medical history for analysis.

There has thus been outlined, rather broadly, the more important features of the disclosure in order that the detailed description thereof that follows may be better understood, and in order that the present contribution to the art may be better appreciated. There are additional features of the disclosure that will be described hereinafter and which will form the subject matter of the claims appended hereto.

The objects of the disclosure, along with the various features of novelty which characterize the disclosure, are pointed out with particularity in the claims annexed to and forming a part of this disclosure.

### BRIEF DESCRIPTION OF SEVERAL VIEWS OF THE DRAWING(S)

The disclosure will be better understood and objects other than those set forth above will become apparent when consideration is given to the following detailed description thereof. Such description makes reference to the annexed drawings wherein:

FIG. 1 is a flow chart view of a medical record management assembly according to an embodiment of the disclosure.

FIG. 2 is a schematic view of an embodiment of the disclosure.

FIG. 3 is a flow chart view of an embodiment of the disclosure displaying the transfer of medical information.

### DETAILED DESCRIPTION OF THE INVENTION

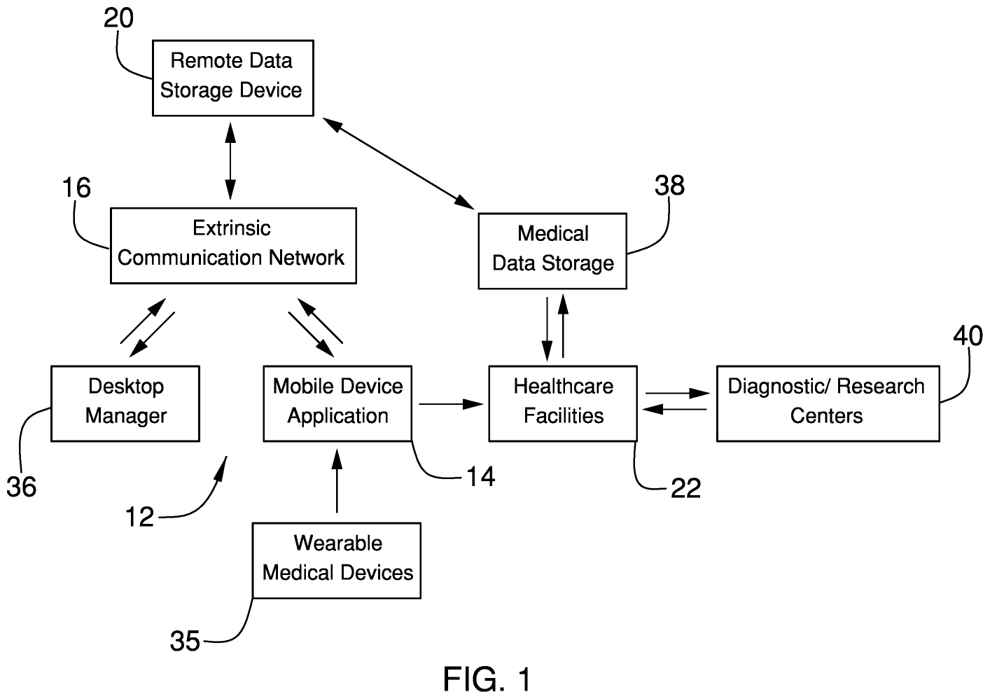

With reference now to the drawings, and in particular to FIGS. 1 through 3 thereof, a new medical record device embodying the principles and concepts of an embodiment of the disclosure and generally designated by the reference numeral 10 will be described.

As best illustrated in FIGS. 1 through 3, the medical record management assembly 10 generally comprises a personal electronic device 12 which stores operational software 14 comprising a medical history of a patient to facilitate the patient to control access to their medical history. The personal electronic device 12 may comprise a smart phone, for example, or other type of electronic device that has wireless communication capabilities and digital data storage capabilities. The personal electronic device 12 is in remote communication with an extrinsic communication network 16, such as the internet or a cellular phone network. The operational software 14 may comprise a smart phone application or other similar type of program.

The operational software 14 performs a data transmission routine when the operational software 14 receives an authorization command. The authorization command may be a digital signature or other type of software based authorization command that is encrypted for security and which is associated with the patient. The operational software 14 transmits selected portions of the medical history to the extrinsic communication network 16 when the operational software 14 receives the authorization command. Additionally, the personal electronic device 12 might include a transceiver that is in wireless communication with the extrinsic communication network 16 and the transceiver may comprise a radio frequency transceiver or the like.

A remote data storage device 20 is included that is in remote communication with the extrinsic communication network 16. In this way the remote data storage device 20 can communicate with the personal electronic device 12 thereby facilitating the medical history to be stored in a centralized location. Thus, the medical history can be easily communicated between a variety of health care providers thereby enhancing accuracy of health care provided for the patient. The remote data storage device 20 may be a data server commonly referred to a cloud server or other type of digital data storage that is accessible via the internet.

The remote data storage device 20 is in remote communication with a plurality of health care systems 22 thereby facilitating the personal electronic device 12 to communicate the selected portions of the medical history to the plurality of health care systems 22. In this way the personal electronic device 12 can facilitate health care professionals to access the selected portions of the medical history for analysis. The remote data storage device 20 receives amendments to the selected portions of the medical history from the plurality of health care systems 22 thereby facilitating the personal electronic device 12 to receive the amendments to the selected portions of the medical history. In this way the patient can maintain continuously updated and accurate medical history on the personal electronic device 12 if the patient so desires.

The plurality of health care systems 22 may include, but not be limited to, a specialty clinic 24, a radiology clinic 26, a general practice clinic 28, a medical laboratory 30 and a physician 32 thereby facilitating the selected portions of the medical history to be amended according to each of the plurality of health care system's 22 specialties. In this way each of the plurality of health care systems 22 can enhance accuracy of the selected portions of the medical history to ensure the best possible medical care for the patient. Additionally, the operational software 14 requires that the patient provides the authorization command before any medical history is released from the remote data storage device 20 to any of the health care systems 22. In this way the data pertaining to the patient's medical history is secure and the patient has control over the security of their medical history.

An artificial intelligence unit 34 is included that is in remote communication with the remote data storage device 20 via the extrinsic communication network 16. The artificial intelligence unit 34 receives selected portions of the medical history from the remote data storage device 20. Furthermore, the artificial intelligence unit 34 can analyze the selected portions of the medical history thereby facilitating the artificial intelligence unit 34 to recommend treatment protocols for the plurality of health care systems 22. In this way the artificial intelligence unit 34 assists the health care professionals to accurately assess the patient's medical needs. Thus, the artificial intelligence unit 34 can reduce unnecessary medical procedures thereby reducing cost of the patient's medical needs. The artificial intelligence unit 34 may comprise proprietary software developed by a software company that is stored and executed on a server, for example, or other type of digital computer.

As is most clearly shown in FIG. 1, wearable medical devices 35, worn by the patient, may be in communication with the personal electronic device 12 to continually update the patient's medical history. A desktop manager 36 may be in remote communication with the remote data storage device 20 for performing administrative duties with respect to the patient's medical history. Furthermore, the remote data storage device 20 may be in remote communication with a medical data storage device 38 maintained at a health care provider, such as a hospital for example. The health care provider is subsequently facilitated to communicate pertinent medical history to diagnostic and research centers 40 for analyzing the medical history of the patient.

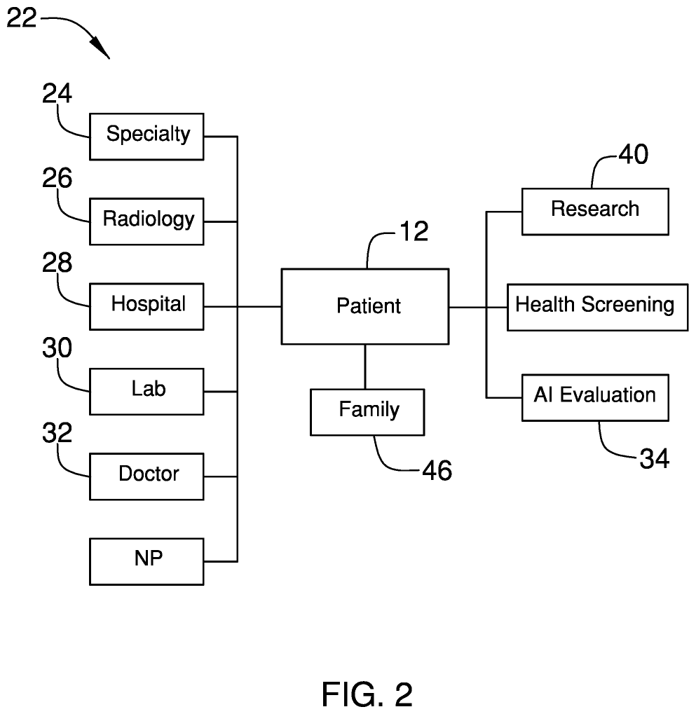

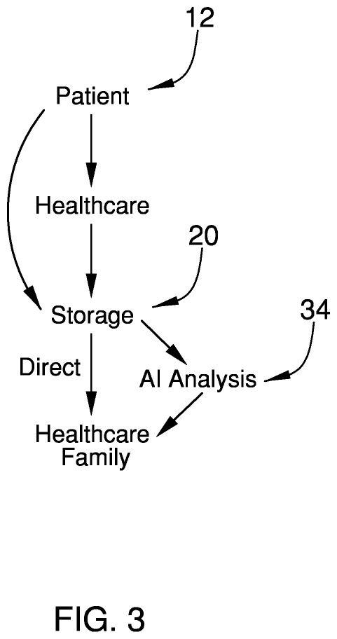

As is most clearly shown in FIG. 2, the personal electronic device 12 can serve as an intermediary between the various health care systems 22 and research facilities 42, health screening services 44 and the artificial intelligence unit 34 to ensure security of the patient's medical history. A flow chart, as is most clearly shown in FIG. 3, demonstrates how information contained in the patient's medical history might be communicated between the patient and various entities involved in the health care industry. Furthermore, the patient can facilitate their family members 46 to access their medical records should the patient decide this is necessary.

In use, the patient's medical history is stored in a centralized location on the remote data storage device 20 which is continuously accessible by the patient via the personal electronic device 12. The patient employs the operational software 14 on the personal electronic device 12 to authorize the release of medical history to various health care systems 22 as well as to authorize amendments to the patient's medical history that are received from the various health care systems 22. In this way the patient's medical history is centralized with respect to the patient and the various health care systems 22 to ensure that the medical history is accurate and is instantly accessible to the patient and the various health care systems 22. The artificial intelligence unit 34 can analyze the patient's medical history to recommend prescriptions, to perform quality control, to recommend health care procedures, to analyze outcome studies and to perform epidemiology studies. In this way the overall cost of the patient's health care can be reduced and the accuracy of the patient's health care can be increased.

With respect to the above description then, it is to be realized that the optimum dimensional relationships for the parts of an embodiment enabled by the disclosure, to include variations in size, materials, shape, form, function and manner of operation, assembly and use, are deemed readily apparent and obvious to one skilled in the art, and all equivalent relationships to those illustrated in the drawings and described in the specification are intended to be encompassed by an embodiment of the disclosure.

Therefore, the foregoing is considered as illustrative only of the principles of the disclosure. Further, since numerous modifications and changes will readily occur to those skilled in the art, it is not desired to limit the disclosure to the exact construction and operation shown and described, and accordingly, all suitable modifications and equivalents may be resorted to, falling within the scope of the disclosure. In this patent document, the word “comprising” is used in its non-limiting sense to mean that items following the word are included, but items not specifically mentioned are not excluded. A reference to an element by the indefinite article “a” does not exclude the possibility that more than one of the element is present, unless the context clearly requires that there be only one of the elements.

I claim:

1. A medical record management assembly for facilitating a patient to control access to their medical records, said assembly comprising:
a personal electronic device storing operational software comprising a medical history of a patient wherein said personal electronic device is configured to facilitate the patient to control access to their medical history, said personal electronic device being in remote communication with an extrinsic communication network;
a remote data storage device being in remote communication with the extrinsic communication network thereby facilitating said remote data storage device to communicate with said personal electronic device thereby facilitating said medical history to be stored in a centralized location, said remote data storage device being in remote communication with a plurality of health care systems thereby facilitating said personal electronic device to communicate said selected portions of said medical history to the plurality of health care systems wherein said personal electronic device is configured to facilitate health care professionals to access said selected portions of said medical history for analysis.

2. The assembly according to claim 1, wherein said operational software performs a data transmission routine when said operational software receives an authorization command, said operational software transmitting selected portions of said medical history to the extrinsic communication network when said operational software receives said authorization command.

3. The assembly according to claim 1, wherein said remote data storage device receives amendments to said selected portions of said medical history from said plurality of health care systems thereby facilitating said personal electronic device to receive said amendments to said selected portions of said medical history.

4. The assembly according to claim 3, wherein said plurality of health care systems includes a specialty clinic and a radiology clinic and a general practice clinic and a medical laboratory and a physician thereby facilitating said selected portions of said medical history to be amended according to each of said plurality of health care systems specialties wherein each of said plurality of health care systems is configured to enhance accuracy of said selected portions of said medical history to ensure the best possible medical care for the patient.

5. The assembly according to claim 1, further comprising an artificial intelligence unit being in remote communication with said remote data storage unit via the extrinsic communication network.

6. The assembly according to claim 5, wherein said artificial intelligence unit receives selected portions of said medical history from said remote data storage unit thereby facilitating said artificial intelligence unit to analyze said selected portions of said medical history thereby facilitating said artificial intelligence unit to recommend treatment protocols for said plurality of health care systems wherein said artificial intelligence unit is configured to assist the health care professionals to accurately assess the patient's medical needs wherein said artificial intelligence unit is configured to reduce unnecessary medical procedures thereby reducing cost of the patient's medical needs.

7. A medical record management assembly for facilitating a patient to control access to their medical records, said assembly comprising:
a personal electronic device storing operational software comprising a medical history of a patient wherein said personal electronic device is configured to facilitate the patient to control access to their medical history, said personal electronic device being in remote communication with an extrinsic communication network, said operational software performing a data transmission routine when said operational software receives an authorization command, said operational software transmitting selected portions of said medical history to the extrinsic communication network when said operational software receives said authorization command, said personal electronic device having a transceiver being in wireless communication with the extrinsic communication network;
a remote data storage device being in remote communication with the extrinsic communication network thereby facilitating said remote data storage device to communicate with said personal electronic device thereby facilitating said medical history to be stored in a centralized location, said remote data storage device being in remote communication with a plurality of health care systems thereby facilitating said personal electronic device to communicate said selected portions of said medical history to the plurality of health care systems wherein said personal electronic device is configured to facilitate health care professionals to access said selected portions of said medical history for analysis, said remote data storage device receiving amendments to said selected portions of said medical history from said plurality of health care systems thereby facilitating said personal electronic device to receive said amendments to said selected portions of said medical history, said plurality of health care systems including a specialty clinic and a radiology clinic and a general practice clinic and a medical laboratory and a physician thereby facilitating said selected portions of said medical history to be amended according to each of said plurality of health care systems specialties wherein each of said plurality of health care systems is configured to enhance accuracy of said selected portions of said medical history to ensure the best possible medical care for the patient; and
an artificial intelligence unit being in remote communication with said remote data storage unit via the extrinsic communication network, said artificial intelligence unit receiving selected portions of said medical history from said remote data storage unit thereby facilitating said artificial intelligence unit to analyze said selected portions of said medical history thereby facilitating said artificial intelligence unit to recommend treatment protocols for said plurality of health care systems wherein said artificial intelligence unit is configured to assist the health care professionals to accurately assess the patient's medical needs wherein said artificial intelligence unit is configured to reduce unnecessary medical procedures thereby reducing cost of the patient's medical needs.
